(19) United States
(12) Patent Application Publication
(10) Pub. No.: US 2015/0342461 A1
(43) Pub. Date: Dec. 3, 2015

(54) OPTICAL BIOMETRIC DEVICE AND POSITION MEASURING DEVICE USED THEREIN

(71) Applicant: SHIMADZU CORPORATION, Kyoto-city, Kyoto (JP)

(72) Inventors: Akihiro ISHIKAWA, Kyoto (JP); Shumpei YAMAGUCHI, Kyoto (JP)

(21) Appl. No.: 14/442,690

(22) PCT Filed: Nov. 14, 2012
(86) PCT No.: PCT/JP2012/079450

§ 371 Date: May 13, 2015

(51) Int. Cl. A61B 5/00; A61B 5/05

(52) U.S. Cl. CPC A61B 5/0042; A61B 5/0059; A61B 5/7445; A61B 5/05; A61B 5/7425; A61B 2560/0475; A61B 2562/0223; A61B 2562/0233

(57) ABSTRACT

The optical biometric device according to the invention is provided with: a measurement data display control unit for displaying a number of pieces of measurement data on the three-dimensional head surface image or three-dimensional brain surface image displayed on the display unit, and is formed of: a storage unit for storing channel information that indicates combinations of light sending probes and light receiving probes for acquiring information about the amounts of the received light in measurement portions; and a channel information display control unit for displaying a number of light sending probe points at which light sending probes have been placed and a number of light receiving probe points at which a number of light receiving probes have been placed according to the three-dimensional coordinates displayed on the display unit, and at the same time, for displaying line segments that indicate combinations of light sending probes and light receiving probes for connecting light sending probe points and light receiving probe points based on said channel information.

### TECHNICAL FIELD

The present invention relates to an optical biometric device and a position measuring device used therein, and in particular, to an optical biometric device for non-invasively measuring brain activity.

### BACKGROUND ART

In recent years, optical imaging devices for simply and non-invasively measuring brain functions using light have been developed in order to observe the state of brain activity. In these optical imaging devices for measuring the brain functions, light sending probes placed on the surface of the head of a subject irradiate the brain with near-infrared rays having three different wavelengths: λ₁, λ₂ and λ₃ (780 nm, 805 nm and 830 nm, for example), and at the same time, light receiving probes placed on the surface of the head detect changes in the intensity of the near-infrared rays (information about the amount of received light) ΔA(λ₁), ΔA(λ₂) and ΔA(λ₃) of the respective wavelengths λ₁, λ₂ and λ₃ emitted from the brain.

In order to find the product of the change in the concentration of the oxyhemoglobin in the blood flow in the brain and the length of the optical path [oxyHb] and the product of the change in the concentration of the deoxyhemoglobin and the length of the optical path [deoxyHb] from the thus-obtained information on the amounts of received light ΔA(λ₁), ΔA(λ₂) and ΔA(λ₃), simultaneous equations (1) to (3) are created using the modified Beer-Lambert Law, for example, and the simultaneous equations are solved. Furthermore, the product of the change in the concentration of the total amount of hemoglobin and the length of the optical path ([oxyHb]+[deoxyHb]) is calculated from the product of the change in the concentration of oxyhemoglobin and the length of the optical path [oxyHb] and the product of the change in the concentration of deoxyhemoglobin and the length of the optical path [deoxyHb].

ΔA(λ₁)=E_{O}(λ₁)×[oxyHb]+E_{d}(λ₁)×[deoxyHb] (1)

ΔA(λ₂)=E_{O}(λ₂)×[oxyHb]+E_{d}(λ₂)×[deoxyHb] (2)

ΔA(λ₃)=E_{O}(λ₃)×[oxyHb]+E_{d}(λ₃)×[deoxyHb] (3)

Here, E_{O}(λₘ) is the absorbance coefficient of oxyhemoglobin for light having a wavelength λₘ, and E_{d}(λₘ) is the absorbance coefficient of deoxyhemoglobin for light having a wavelength λₘ.

Here, the relationship between the distance between a light-transmitting probe and a light-receiving probe and the portion to be measured is described. FIG. 7 is a diagram showing the relationship between a pair of probes, a light-transmitting probe and a light-receiving probe, and the portion to be measured. A light-transmitting probe 12 is pressed against a light transmitting point T on the surface of the head of a subject, and at the same time, a light-receiving probe 13 is pressed against a light receiving point R on the surface of the head of the subject. Thus, light is emitted from the light-transmitting probe 12, and at the same time, the light released from the surface of the head enters into the light-receiving probe 13. At this time, the light that has passed through the banana-shaped area (area to be measured) from among the light emitted from the light transmitting point T on the surface of the head reaches the light receiving point R on the surface of the head. As a result, information on the amount of received light A (λ₁), A (λ₂) and A (λ₃) concerning the portion to be measured S of the subject at a depth, which is half of the distance along the line connecting the light transmitting point T and the light receiving point R along the surface of the head of the subject from the mid-point M of the line connecting the light transmitting point T and the light receiving point R along the surface of the head of the subject, is particularly gained from among the area to be measured.

In optical brain function imaging devices, the product [oxyHb] of the change in the concentration of oxyhemoglobin and the length of the light path, the product [deoxyHb] of the change in the concentration of deoxyhemoglobin and the length of the light path, and the product ([oxyHb]+[deoxyHb]) of the change in the concentration of total hemoglobin and the length of the light path concerning a number of portions to be measured in the brain are measured.

In such optical brain function imaging devices, a holder (light sending/receiving unit) is used so as to provide holder units in a grid-like form for holding light sending probes 12_{T1} through 12_{T8} and light receiving probes 13_{R1} through 13_{R8}, which are to be placed on the surface of the head of a subject in order to make the eight light sending probes and the eight light receiving probes make contact with the surface of the head in a predetermined alignment. At the same time, the holder units are linked to each other through flexible linking portions and the linking portions are rotatable within a predetermined angle with the holder units as rotational axes (see Patent Document 1).

FIG. 2 is a plan diagram showing an example of a holder into which eight light sending probes and eight light receiving probes are to be inserted. The light sending probes 12_{T1} through 12_{T8} and the light receiving probes 13_{R1} through 13_{R8} are put in place through alternate insertion in a matrix of four probes in the longitudinal direction and four probes in the lateral direction. At this time, the intervals between the light sending probes 12_{T1} to 12_{T8} and the light receiving probes 13_{R1} to 13_{R8} are 30 mm Here, different numbers (T1, T2 . . . , R1, R2 . . . ) are allocated to through holes in the holder 30 so that it can be recognized which light sending probe 12_{T1} to 12_{T8} or light receiving probe 13_{R1} to 13_{R8} has been inserted into which through hole, and at the same time, different numbers (T1, T2 . . . ) are allocated to light sending probes 12_{T1} to 12_{T8}, and different numbers (R1, R2 . . . ) are allocated to light receiving probe 13_{R1} to 13_{R8}, respectively. As a result, information on the amounts of light ΔAₙ(λ₁), ΔAₙ(λ₂) and ΔAₙ(λ₃) (n=1, 2, 3 . . . , 24) received from the 24 portions in the brain is obtained.

Thus, the 24 pieces of information about the amount of received light ΔAₙ(λ₁), ΔAₙ(λ₂) and ΔAₙ(λ₃) are gained at predetermined time intervals Δt so that the chronological change (measurement data) Xₙ(t) in the product [oxyHb] of the change in the concentration of oxyhemoglobin and the length of the light path, the chronological change (measurement data) Yₙ(t) in the product [deoxyHb] of the change in the concentration of deoxyhemoglobin and the length of the light path, and the chronological change (measurement data) Zₙ(t) in the product ([oxyHb]+[deoxyHb]) of the change in the concentration of total hemoglobin and the length of the light path can be found using the relational expressions (1), (2) and (3) (n=1, 2 . . . , 24).

In addition, the chronological change (measurement data) Xₙ(t) in the product [oxyHb] of the change in the concentration of oxyhemoglobin and the length of the light path and other data are displayed on the display unit as images that a doctor, or the like, may observe. For example, the chronological change (measurement data) Xₙ(t₁) in the product [oxyHb] of the change in the concentration of oxyhemoglobin and the length of the light path that is gained from 24 portions in total on the surface of the brain at a certain point in time t₁ is displayed through color mapping on the basis of a color table that indicates the correspondence between numeric values and colors. At this time, a doctor, or the like, must recognize from which portion of the brain the chronological change (measurement data) Xₙ(t₁) in the product [oxyHb] of the change in the concentration of oxyhemoglobin and the length of the light path has been gained, because the anatomical structure of the brain differs according to individual and individuals have differing shapes of brain. In order to do so, three-dimensional image data showing the surface of the brain of a patient is gained from a magnetic resonance imaging diagnostic device (hereinafter abbreviated as MRI) so as to display a three-dimensional brain surface image and, thus, the chronological change (measurement data) Xₙ(t₁) in the product [oxyHb] of the change in the concentration of oxyhemoglobin and the length of the light path is displayed through color mapping, which is superposed on the three-dimensional brain surface image (see Patent Document 2). FIG. 6 is a diagram showing an example of a display screen that displays 24 pieces of measurement data Xₙ(t₁) through color mapping.

In order to superpose the chronological change (measurement data) Xₙ(t₁) in the product [oxyHb] of the change in the concentration of oxyhemoglobin and the length of the light path on the three-dimensional brain surface image 42 for display, it is necessary to designate the points at which the light sending probes 12_{T1} through 12_{T8} and the light receiving probes 13_{R1} through 13_{R8} are placed on the three-dimensional brain surface image 42. FIGS. 3 and 4 are diagrams illustrating a method for diagnosing the points at which the light sending probes 12_{T1} through 12_{T8} and the light receiving probes 13_{R1} through 13_{R8} are placed. FIG. 3 is a diagram showing an example of a three-dimensional image displayed on the display screen of an optical biometric device. FIG. 4 is a diagram showing the relationship between a holder 30 placed on the head of a subject, a magnetic field source 14 fixed to a set point (on the lower jaw of the subject), and a stylus 15 in the form of a pencil that is manipulated by a doctor, a clinical examination technician or the like.

As shown in FIG. 4, the magnetic field source 14 for generating a magnetic field in a space including and surrounding the head of the patient is fixed to the lower jaw or the like of the patient, and a doctor, a clinical examination technician or the like uses the stylus 15 having a magnetic sensor for designation in an end portion 15a with which the positional relationship vis-à-vis the magnetic source 14 can be detected so as to designate three standard points (base of the nose B1, left auricle B2, right auricle, for example) on the surface of the head of the patient. In addition, as shown in FIG. 3, three standard point images (image of base of the nose, image of left auricle, image of right auricle B3G, for example) that correspond to the three standard points B1, B2 are designated on the three-dimensional head surface image 41 displayed on the display unit using a pointer 43. As a result, the surface of the head of the subject and the surface of the brain are compared with the three-dimensional head surface image 41 and the three-dimensional brain surface image 42. After that, the stylus 15 is used to sequentially designate (in ascending or descending order) the points at which the light sending probes 12_{T1} through 12_{T8} and the light receiving probes 13_{R1} through 13_{R8} are placed on the surface of the head of the subject so that the points at which the light sending probes 12_{T1} through 12_{T8} and the light receiving probes 13_{R1} through 13_{R8} are placed are indicated on the three-dimensional brain surface image 42.

In order to confirm the inputted points at which the light sending probes 12_{T1} through 12_{T8} and the light receiving probes 13_{R1} through 13_{R8} have been placed, three-dimensional coordinates (XYZ coordinates) with the magnetic field source 14 as the original point are displayed on the display screen so that the points at which the light sending probes 12_{T1} through 12_{T8} and the light receiving probes 13_{R1} through 13_{R8} have been placed are displayed according to the XYZ coordinates. FIG. 8 shows an example of a display screen for confirming the inputted points at which the light sending probes 12_{T1} through 12_{T8} and the light receiving probes 13_{R1} through 13_{R8} have been placed. In the right region of the display screen the point at which each light sending probe 12_{T1} through 12_{T8} is placed is displayed according to the XYZ coordinates as a red globe with a corresponding number in such a manner that the point at which the light sending probe 12_{T1} is placed is displayed as a red globe with the number 1 as the light sending probe location point T1, and the point at which the light sending probe 12_{T2} is placed is displayed as a red globe with the number 2 as the light sending probe location point T2. In addition, the point at which each light receiving probe 13_{R1} through 13_{R8} is placed is displayed according to the XYZ coordinates as a blue globe with a corresponding number in such a manner that the point at which the light receiving probe 13_{R1} is placed is displayed as a blue globe with the number 1 as the light receiving probe location point R1, and the point at which the light sending probe 13_{R2} is placed is displayed as a blue globe with the number 2 as the light receiving probe location point R2.

Here, the coordinates (X, Y, Z) of the point at which each light receiving probe 13_{R1} through 13_{R8} is placed are shown in the lower left region of the display screen.

### PRIOR ART DOCUMENT

### Patent Document

Patent Document 1: Japanese Unexamined Patent Publication 2002-143169
Patent Document 2: Japanese Unexamined Patent Publication 2009-172177

### SUMMARY OF THE INVENTION

### Problem to be Solved by the Invention

In the case wherein a display screen as in FIG. 8 is displayed in order to confirm the inputted points at which the light sending probes 12_{T1} through 12_{T8} and the light receiving probes 13_{R1} through 13_{R8} have been placed, it is difficult for a doctor, a clinical examination technician or the like to determine whether or not the points at which the light sending probes 12_{T1} through 12_{T8} and the light receiving probes 13_{R1} through 13_{R8} are to be placed have been inputted in the correct order even though all of the points at which the light sending probes 12_{T1} through 12_{T8} and the light receiving probes 13_{R1} through 13_{R8} are to be placed have been inputted when viewing the display screen as in FIG. 8.

Therefore, an object of the present invention is to provide an optical biometric device and a position measuring device used therein with which it can be easily determined whether or not the points at which the light sending probes 12_{T1} through 12_{T8} and the light receiving probes 13_{R1} through 13_{R8} are to be placed have been inputted correctly.

### Means for Solving Problem

In order to achieve the above described object, the optical biometric device according to the present invention is provided with: a light sending/receiving unit having a number of light sending probes to be placed on a surface of the head of a subject and a number of light receiving probes to be placed on a surface of the head; a control unit for sending and receiving light which acquires a number of pieces of information about the amount of light received in a number of measurement portions under control such that the above described light sending probes irradiate the surface of the head with light, and at the same time the above described light receiving probes detect light emitted from the surface of the head; an operation unit for acquiring a number of pieces of measurement data on the basis of a number of pieces of information about the amount of received light; a three-dimensional image display control unit for acquiring a three-dimensional head surface image and a three-dimensional brain surface image and displaying the acquired images on a display unit; and a measurement data display control unit for displaying a number of pieces of measurement data on the three-dimensional head surface image or three-dimensional brain surface image displayed on the display unit, and is characterized by further having: a storage unit for storing channel information that indicates combinations of light sending probes and light receiving probes for acquiring information about the amounts of the received light in measurement portions; and a channel information display control unit for displaying a number of light sending probe points at which light sending probes have been placed and a number of light receiving probe points at which a number of light receiving probes have been placed according to the three-dimensional coordinates displayed on the display unit, and at the same time, for displaying line segments that indicate combinations of light sending probes and light receiving probes for connecting light sending probe points and light receiving probe points based on the above described channel information.

Here, the “measurement data” may be the chronological change in the information about the amount of received light that has been detected by light receiving probes or may be the chronological change in the concentration of the oxyhemoglobin calculated from the information about the amount of received light, the chronological change in the concentration of deoxyhemoglobin or the chronological change in the concentration of the total hemoglobin.

In addition, the “three-dimensional head surface image” means a three-dimensional image that has been prepared from the video data of a subject created through MRI or CT imaging by sampling video data showing the surface of the head or a three-dimensional head surface template that shows the surface of a standard three-dimensional head. Furthermore, the “three-dimensional brain surface image” means a three-dimensional image that has been prepared from the video data of a subject created through MRI or CT imaging by sampling video data showing the surface of the brain or a three-dimensional brain surface template that shows the surface of a standard three-dimensional brain.

### Effects of the Invention

In the optical biometric device according to the present invention, the display unit displays line segments connecting light sending probe points and light receiving probe points according to three-dimensional coordinates and, therefore, a doctor, a clinical examination technician or the like can confirm whether or not line segments are aligned in a grid-like form matching that of the light sending/receiving units and, thus, can easily determine whether or not the points at which light sending probes are to be placed and the points at which light receiving probes are to be placed have been inputted correctly.

(Other Means for Solving Problem and Effects Thereof)

Alternatively, the Optical Biometric Device According to the Present Invention May further be provided with: a magnetic field source for generating a magnetic field in a space including and surrounding the head of the above described subject that is fixed to a set point on the head of the above described subject; a magnetic sensor for designation that detects a magnetic field in order to designate a point on the surface of the head of the above described subject; a standard positional relationship acquisition unit for acquiring the positional relationship between the above described magnetic field source and at least three standard points by gaining a detection signal from the above described magnetic sensor for designation when the three standard points are designated on the surface of the head of the above described subject by the magnetic sensor for designation; a correspondence data preparation unit for preparing correspondence data that indicates the correspondence between the three standard points and at least three standard point images when the three standard point images are designated on the above described three-dimensional head surface image by an input unit; and a placed point positional relationship acquisition unit for acquiring positional relationships between the above described magnetic source and the points at which the light sending probes and the light receiving probes are placed by gaining a detection signal from the above described magnetic sensor for designation when the points at which the light sending probes are placed and the points at which the light receiving probes are placed on the surface of the head of the above described subject are designated by the magnetic sensor for designation.

Here, the “magnetic sensor for designation” is used to designate standard points (base of nose, left auricle, right auricle, for example) on the surface of the head of a subject, or to designate the points at which light sending probes are placed and the points at which light receiving probes are placed, and a stylus in a rod form having a magnetic sensor for designation in an end portion can be cited as an example.

In addition, the “set point on the head of a subject” is any point at which the magnetic field source could generate a magnetic field in a space including and surrounding the head of the subject, and a point on the lower jaw can be cited as an example.

Furthermore, the position measuring device according to the present invention is used in an optical biometric device having: a light sending/receiving unit having a number of light sending probes to be placed on a surface of the head of a subject and a number of light receiving probes to be placed on a surface of the head; and a control unit for sending and receiving light which acquires a number of pieces of information about the amount of light received in a number of measurement portions under control such that the above described light sending probes irradiate the surface of the head with light, and at the same time the above described light receiving probes detect light emitted from the surface of the head, and is characterized by having: a storage unit for storing channel information that indicates combinations of light sending probes and light receiving probes for acquiring information about the amounts of the received light in measurement portions; and a channel information display control unit for displaying a number of light sending probe points at which light sending probes have been placed and a number of light receiving probe points at which a number of light receiving probes have been placed according to the three-dimensional coordinates displayed on a display unit, and at the same time, for displaying line segments that indicate combinations of light sending probes and light receiving probes for connecting light sending probe points and light receiving probe points based on the above described channel information.

### BRIEF DESCRIPTION OF THE DRAWINGS

FIG. 1 is a block diagram showing the configuration of the optical biometric device according to one embodiment of the present invention;

FIG. 2 is a plan diagram showing an example of a holder into which eight light sending probes and eight light receiving probes are to be inserted;

FIG. 3 is a diagram showing an example of a display screen that displays a three-dimensional image;

FIG. 4 is a diagram showing the relationship between the holder mounted on the head of the subject, a magnetic field source fixed to a set point, and a stylus utilized by a doctor, or the like;

FIG. 5 is a diagram showing another example of a display screen for confirming the inputted points at which light sending probes and light receiving probes have been placed;

FIG. 6 is a diagram showing an example of a display screen that displays 24 pieces of measurement data Xₙ(t₁) through color mapping;

FIG. 7 is a diagram showing the relationship between a measurement portion and a pair of probes, a light sending probe and a light receiving probe; and

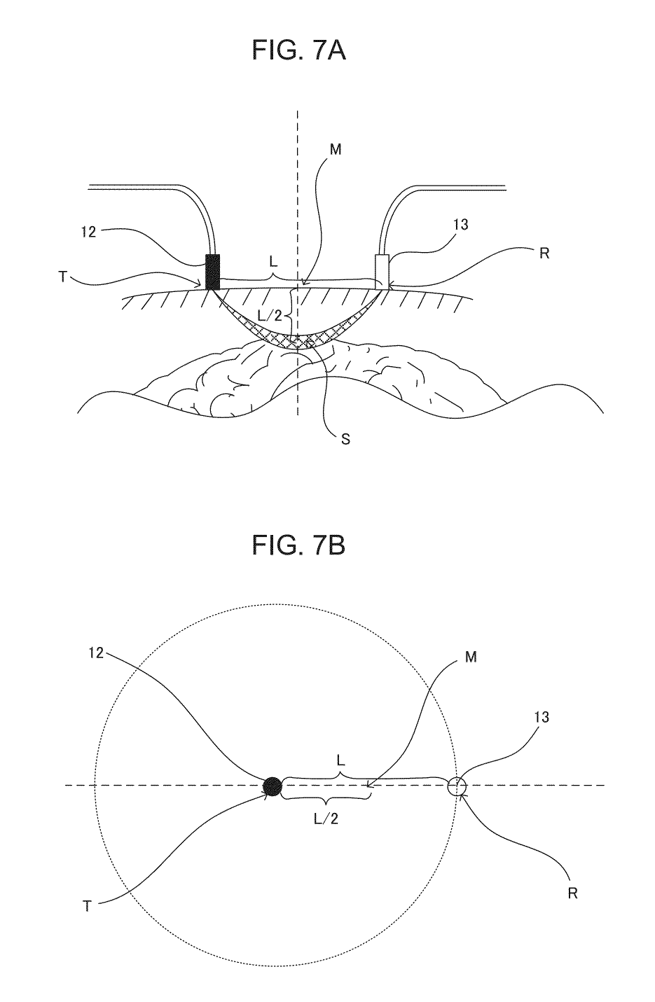

FIG. 8 is an example of a display screen for confirming the inputted points at which light sending probes and light receiving probes have been placed.

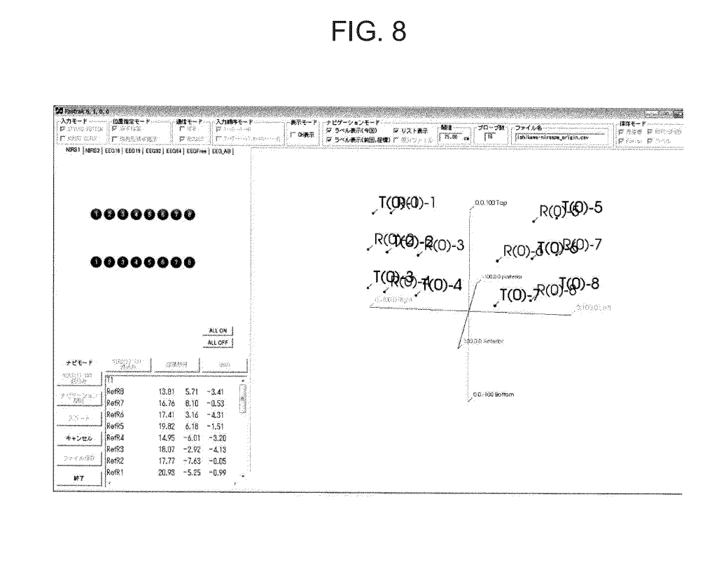

### PREFERRED EMBODIMENTS OF THE INVENTION

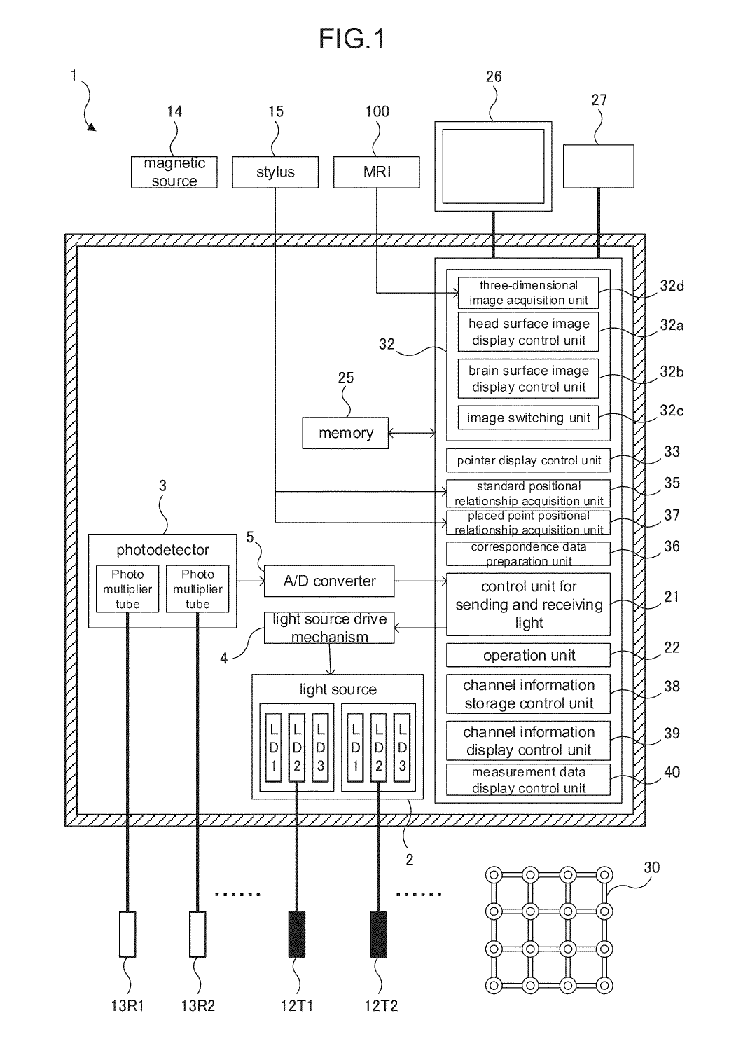

In the following the preferred embodiments of the present invention are described in reference to FIGS. 1 through 6. Here, the present invention is not limited to the below described embodiments but includes various modifications as long as the gist of the present invention is not deviated from.

FIG. 1 is a block diagram showing the configuration of the optical biometric device according to one embodiment of the present invention.

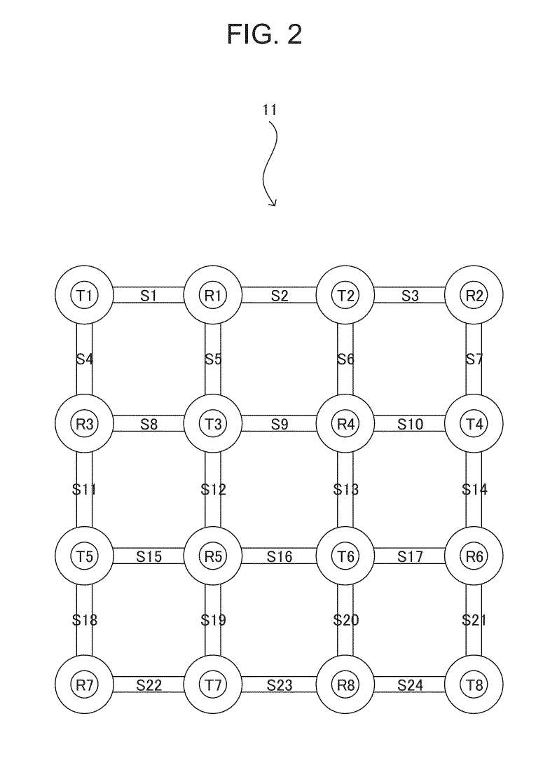

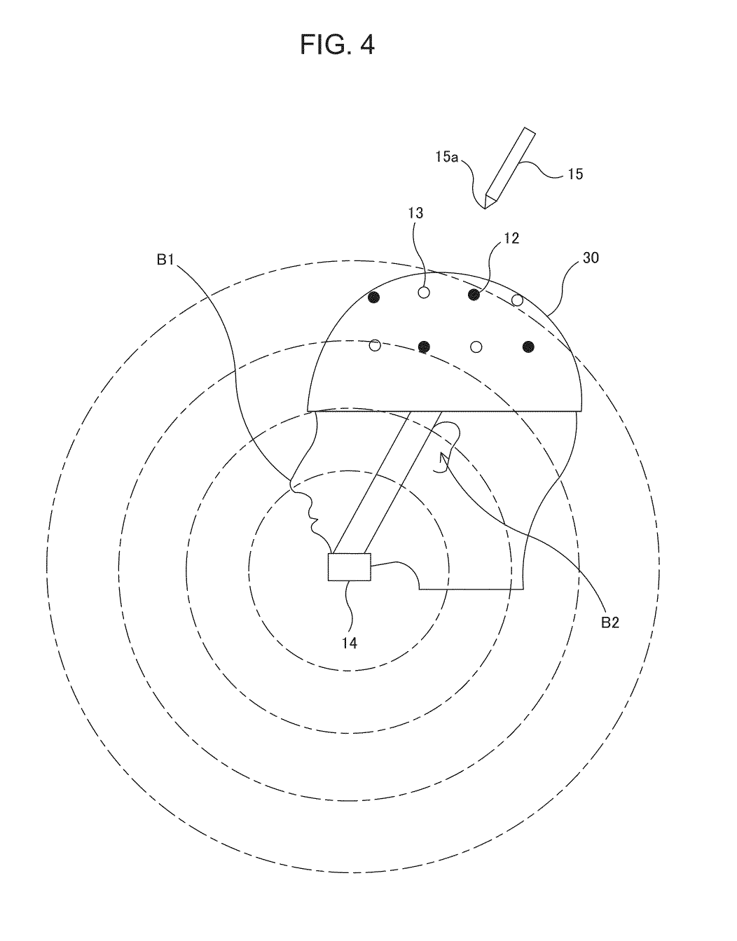

An optical biometric device 1 is provided with a light source 2 for emitting light, a light source drive mechanism 4 for driving the light source 2, a photodetector 3 for detecting light, an A/D converter 5, a control unit 21 for sending and receiving light, an operation unit 22, a three-dimensional image display control unit 32, a pointer display control unit 33, a standard positional relationship acquisition unit 35, a correspondence data preparation unit 36, a placed point positional relationship acquisition unit 37, a channel information storage control unit 38, a channel information display control unit 39, a measurement data display control unit 40, and a memory 25, and is also provided with eight light sending probes 12T1 through 12T8 as well as eight light receiving probes 13R1 through 13R8 as in FIG. 2, a display unit 26, an input unit 27, a holder (light sending/receiving unit) 30, a magnetic field source 14 for generating an alternating magnetic field in a space including and surrounding the head of a subject, and a stylus 15 in a rod form having a magnetic sensor 15a for designation in an end portion in order to detect an alternating magnetic field as in FIG. 4.

The light source drive mechanism 4 drives the light source 2 in response to a drive signal inputted from the control unit 21 for sending and receiving light. The light source 2 consists of semiconductor lasers LD1, LD2, LD3 and the like that can emit near-infrared rays having three different wavelengths λ₁, λ₂ and λ₃ for example.

The photodetector 3 consists of a photomultiplier tube or the like and individually detects near-infrared rays received by the eight light receiving probes 13R1 through 13R8 so as to output eight pieces of information about the amount of received light ΔA(λ₁), ΔA(λ₂) and ΔA(λ₃) to the control unit 21 for sending and receiving light via the A/D converter 5.

The three-dimensional image display control unit 32 has a three-dimensional image acquisition unit 32d, a head surface image display control unit 32a, a brain surface image display control unit 32b and an image switching unit 32c.

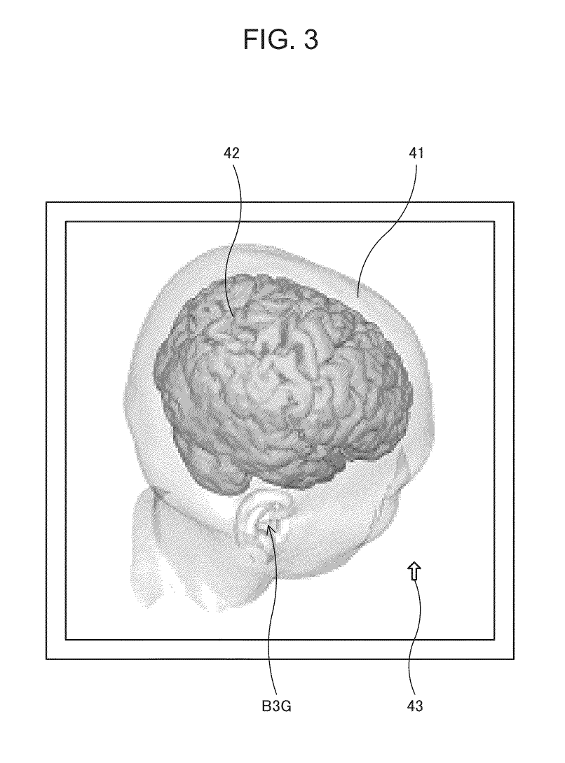

The three-dimensional image acquisition unit 32d acquires video data that has been prepared by MRI 100 before measurement and, thus, acquires three-dimensional head surface image data by sampling video data showing the surface of the head and, at the same time, acquires three-dimensional brain surface image data by sampling video data showing the surface of the brain so as to carry out such control that the three-dimensional head surface image data and the three-dimensional brain surface image data are stored in the memory 25. Here, the MRI 100 prepares video data showing three-dimensional images in three directions. Here, the displayed video data shows a subject including the surface of the head and the surface of the brain as in FIG. 3. In addition, video data is formed of a number of pixels having numerical values of the intensity information, the phase information and the like of the MRI signal. As examples of the above described sampling method, a region expansion method, a region annexation method, an image region division method such as a heuristic method, a method for sampling a region by linking border elements and a method for sampling a region by changing the forms of closed curves can be cited when using a number of pixels having numerical values of the intensity information, the phase information, and the like of the MRI signal.

The head surface image display control unit 32a carries out such control that a head surface image 41 is displayed on the display unit 26 based on the three-dimensional head surface image data stored in the memory 25 (see FIG. 3). Here, a doctor, a clinical examination technician or the like can use the input unit 27 so that the direction in which the displayed three-dimensional head surface image 41 is viewed can be changed to the desired direction. In addition, the three-dimensional head surface image 41 can be displayed in a translucent manner or in color.

The brain surface image display control unit 32b carries out such control that the three-dimensional brain surface image 42 is displayed on the display unit 26 based on the three-dimensional brain surface video data stored in the memory 25. Here, a doctor, a clinical examination technician or the like can use the input unit 27 so that the direction in which the displayed three-dimensional brain surface image 42 is viewed can be changed to the desired direction.

The image switching unit 32c carries out such control that the three-dimensional head surface image 41 is determined to be displayed in the head surface image display control unit 32a, the three-dimensional brain surface image 42 is determined to be displayed in the brain surface image display control unit 32b, and the three-dimensional head surface image 41 is determined to be displayed in the head surface image display control unit 32a and, at the same time, the three-dimensional brain surface image 42 is determined to be displayed in the brain surface image display control unit 32b. In the case wherein the three-dimensional head surface image 41 can be displayed in the head surface image display control unit 32a and, at the same time, the three-dimensional brain surface image 42 can be displayed in the brain surface image display control unit 32b, the three-dimensional head surface image 41 and the three-dimensional brain surface image 42 are displayed in such a state that their positions are overlapping.

The pointer display control unit 33 displays a pointer 43 on the display unit 26 and, at the same time, carries out such control that the pointer 43 displayed on the display unit 26 is shifted or a point on the image is designated using the pointer 43 on the basis of an operation signal outputted form the input device 27.

The magnetic field source 14 in FIG. 4 is formed of a solenoid coil where a wire coated with an insulator is wound around a hard insulating columnar core, for example, and generates an alternating magnetic field. In addition, the magnetic field source 14 is fixed to a set point (on the lower jaw of the subject in the present embodiment) so that the alternating magnetic field is generated in a space including and surrounding the head of the subject.

In addition, the stylus 15 is in a rod form and has a magnetic sensor 15a for designation in its end portion. The magnetic sensor 15a for designation has wires that are wound around three axes that are orthogonal to each other so as to form three coils, each of which detects a detection signal with an intensity that is proportional to the intensity of the component of the magnetic field in the direction of the axis of the relevant coil. Thus, a doctor, a clinical examination technician or the like designates three standard points on the surface of the head of the subject (base of the nose B1, left auricle B2, right auricle), points at which eight light sending probes 12_{T1} through 12_{T8} are placed and points at which eight light receiving probes 13_{R1} through 13_{R8} are placed so that detection signals can be out putted to the standard positional relationship acquisition unit 35 and the placed point positional relationship acquisition unit 37.

The standard positional relationship acquisition unit 35 receives a detection signal from the stylus 15 when a doctor, a clinical examination technician, or the like designates three standard points on the surface of the head of the subject (base of the nose B1, left auricle B2, right auricle) with the stylus 15 and thus carries out such control that the positional relationship between the magnetic field source 14 and the three standard points is recognized.

The correspondence data preparation unit 36 carries out such control that correspondence data that indicates the correspondence between the three standard points and the three standard point images is prepared when the three standard point images (image of base of the nose, image of left auricle, image of right auricle B3G) that correspond to the three standard points (base of the nose B1, left auricle B2, right auricle) are designated with the pointer 43 in the three-dimensional head surface image 41 and in the three-dimensional brain surface image 42 as displayed on the display unit 26. That is to say, the head surface and the brain surface of the subject are compared with the three-dimensional head surface image 41 and the three-dimensional brain surface image 42 in the optical biometric device 1.

The placed point positional relationship acquisition unit 37 receives a detection signal from the stylus 15 when a doctor, a clinical examination technician or the like designates points at which the light sending probes 12_{T1} through 12_{T8} and the light sending probes 13_{R1} through 13_{R8} are placed on the surface of the head of the subject and thus carries out such control that the positional relationship between the magnetic field source 14 and the eight light sending probes 12_{T1} through 12_{T8} and the eight light receiving probes 13_{R1} through 13_{R8} is recognized.

Specifically, the doctor, the clinical examination technician or the like places the holder 30 on the surface of the head of the subject, and after that sequentially designates points at which the light sending probes 12_{T1} through 12_{T8} are placed on the surface of the head of the subject and inserts the light sending probes into the corresponding through holes in such a manner that the stylus 15 is used so as to designate one through hole in the holder 30 as a point at which the light sending probe 12_{T1} is placed, and thus the light sending probe 12_{T1} is inserted into this through hole, and then the stylus 15 is used so as to designate another through hole in the holder 30 as a point at which the light sending probe 12_{T2} is placed, and thus the light sending probe 12_{T2} is inserted into this through hole. In addition, points at which the light receiving probes 13_{R1} through 13_{R8} are placed on the surface of the head of the subject are sequentially designated and the light receiving probes are inserted into the corresponding through holes in such a manner that the stylus 15 is used so as to designate one through hole in the holder 30 as a point at which the light receiving probe 13_{R1} is placed, and thus the light receiving probe 13_{R1} is inserted into this through hole, and then the stylus 15 is used so as to designate another through hole in the holder 30 as a point at which the light receiving probe 13_{R2} is placed, and thus the light sending probe 12_{T2} is inserted into this through hole.

As a result, in the optical biometric device 1, the surface of the head and the surface of the brain of the subject are compared with the three-dimensional head surface image 41 and the three-dimensional brain surface image 42 in the correspondence data preparation unit 36, and thus information about the points at which the probes are placed is prepared so as to indicate at which points the light sending probes 12_{T1} through 12_{T8} and the light receiving probes 13_{R1} through 13_{R8} are placed respectively on the three-dimensional head surface image 41 and the three-dimensional brain surface image 42.

The channel information storage control unit 38 carries out such control that channel information ΔAₙ(λ₁), ΔAₙ(λ₂) and ΔAₙ(λ₃) (n=1, 2, 3 . . . , 24) that indicates the combinations of the light sending probes 12_{T1} through 12_{T8} and the light receiving probes 13_{R1} through 13_{R8} in order to acquire information about the amount of light received from measurement portions, of which the number is 24 in total, before measurement is stored in the memory 25. Specifically, the memory 25 stores channel information that indicates channels of a total of 24 pairs of a light sending probe and a light receiving probe for acquiring a total of 24 pieces of information about the amount of received light ΔAₙ(λ₁), ΔAₙ(λ₂) and ΔAₙ(λ₃) (n=1, 2, 3 . . . , 24), each of which is gained when light from a certain light sending probe is detected by a certain light receiving probe in such a manner that information about the amount of received light ΔA₁(λ₁), ΔA₁(λ₂) and ΔA₁(λ₃) is acquired when light from the light sending probe 12_{T1} is detected by the light receiving probe 13_{R1} through the channel between the first pair, and information about the amount of received light ΔA₂(λ₁), ΔA₂(λ₂) and ΔA₂(λ₃) is acquired when light from the light sending probe 12_{T2} is detected by the light receiving probe 13_{R1} through the channel between the second pair.

The channel information display control unit 39 displays three-dimensional coordinates (XYZ coordinates) on the display unit 26 when a doctor, a clinical examination technician or the like uses the input unit 27 to input an operation signal for confirming the inputted points at which the light sending probes 12_{T1} through 12_{T8} and the light receiving probes 13_{R1} and 13_{R8} have been placed. Thus, the channel information display control unit 39 displays, according to the XYZ coordinates, eight light sending probe points T1 through T8 at which eight light sending probes 12_{T1} through 12_{T8} are placed and eight light receiving probe points R1 through R8 at which eight light receiving probes 13_{R1} through 13_{R8} are placed, and carries out such control that lines that connect the light sending probe points T1 through T8 and the light receiving probe points R1 through R8 are displayed on the basis of the channel information stored in the memory 25.

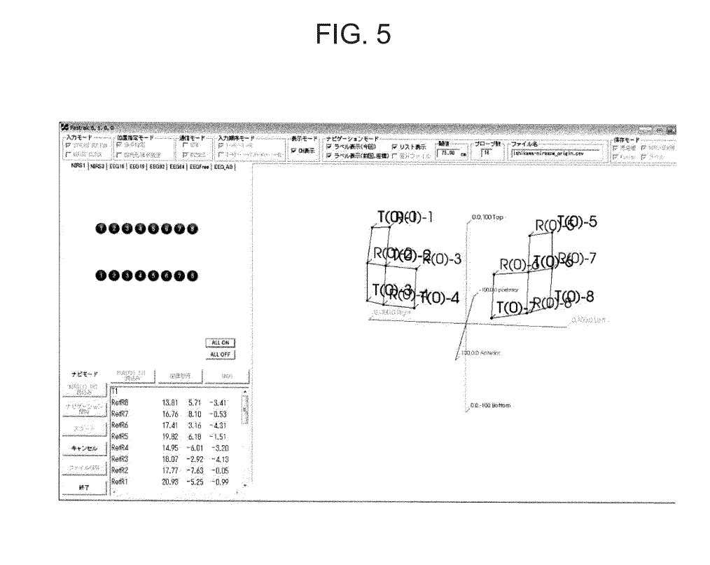

FIG. 5 shows an example of a display screen for confirming the inputted points at which the light sending probes 12_{T1} through 12_{T8} and the light receiving probes 13_{R1} and 13_{R8} have been placed. In the right region of the display screen the point at which each light sending probe 12_{T1} through 12_{T8} is placed is displayed according to the XYZ coordinates as a red globe with a corresponding number in such a manner that the point at which the light sending probe 12_{T1} is placed is displayed as a red globe with the number 1 as the light sending probe location point T1, and the point at which the light sending probe 12_{T2} is placed is displayed as a red globe with the number 2 as the light sending probe location point T2. In addition, the point at which each light receiving probe 13_{R1} through 13_{R8} is placed is displayed according to the XYZ coordinates as a blue globe with a corresponding number in such a manner that the point at which the light receiving probe 13_{R1} is placed is displayed as a blue globe with the number 1 as the light receiving probe location point R1, and the point at which the light sending probe 13_{R2} is placed is displayed as a blue globe with the number 2 as the light receiving probe location point R2. Furthermore, 24 lines that indicate a total of 24 pairs of channels are displayed in such a manner that a line that indicates the channel between the first pair that connects the light sending probe point T1 and the light receiving probe point R1 is displayed, and a line that indicates the channel between the second pair that connects the light sending probe point T2 and the light receiving probe point R1 is displayed.

In the lower left region on the display screen in FIG. 5, the coordinates (X, Y, Z) for the points at which the respective light receiving probes 13_{R1} through 13_{R8} are placed are displayed.

As a result, lines that connect the light sending probe points T1 through T8 and the light receiving probe points R1 through R8 are displayed according to the XYZ coordinates on the display unit 26 and therefore a doctor, a clinical examination technician or the like can confirm on the screen whether or not the lines are arranged in a grid-like form that is the same as that of the holder 30 when confirming the inputted points at which the light sending probes 12_{T1} through 12_{T8} and the light receiving probes 13_{R1} and 13_{R8} have been placed.

In the case where the doctor, the clinical examination technician or the like makes a mistake in the designation of a point when using the stylus 15 to designate the points at which the light sending probes 12_{T1} through 12_{T8} and the light receiving probes 13_{R1} through 13_{R8} are to be placed, the lines displayed on the display unit are not arranged in the same grid-like form as that of the holder 30.

Thus, the doctor, the clinical examination technician or the like can easily determine whether or not the points at which the light sending probes 12_{T1} through 12_{T8} were placed and the points at which the light receiving probes 13_{R1} and 13_{R8} were placed have been inputted correctly. As a result, 24 pieces of measurement data Xₙ(t), Yₙ(t) and Zₙ(t)(n=1, 2 . . . , 24) can be acquired from the correct measurement portions.

The control unit 21 for sending and receiving light outputs a drive signal for sending light to one light sending probe 12_{T1} through 12_{T8} at a predetermined point in time to the light source drive mechanism 4 and at the same time to carries out such control that the photodetector 3 detects the information ΔAₙ(λ1), ΔAₙ(λ₂) and ΔAₙ(λ₃) (n=1, 2, 3 . . . , 24) about the amount of light received by the light receiving probes 13_{R1} and 13_{R8}. Specifically, light is sequentially sent to each light sending probe 12_{T1} through 12_{T8} according to a predetermined timing in such a manner that light with a wavelength of 780 nm is sent to the light sending probe 12_{T1} for the first 5 milliseconds, light with a wavelength of 805 nm is sent to the light sending probe 12_{T1} for the next 5 milliseconds, light with a wavelength of 830 nm is sent to the light sending probe 12_{T1} for the next 5 milliseconds, and light with a wavelength of 780 nm is sent to the light sending probe 12_{T2} for the next 5 milliseconds. Here, information about the amount of received light is detected by the eight light receiving probes 13_{R1} through 13_{R8} whenever light is sent to any one of the light sending probes 12_{T1} through 12_{T8}, and the information about the received light from a predetermined light receiving probe 13_{R1} through 13_{R8} that has been detected according to a predetermined timing is stored in the memory 25 on the basis of the channel information stored in the memory 25. As a result, a total of 24 pieces of information ΔAₙ(λ₁), ΔAₙ(λ₂) and ΔAₙ(λ₃) (n=1, 2, 3 . . . , 24) about the amount of received light are collected.

The operation unit 22 carries out such control that the chronological change (measurement data) Xₙ(t) in the product [oxyHb] of the change in the concentration of oxyhemoglobin and the length of the light path, the chronological change (measurement data) Yₙ(t) in the product [deoxyHb] of the change in the concentration of deoxyhemoglobin and the length of the light path, and the chronological change (measurement data) Zₙ(t) in the product ([oxyHb]+[deoxyHb]) of the change in the concentration of total hemoglobin and the length of the light path are found using the relational expressions (1), (2) and (3) (n=1, 2 . . . , 24) on the basis of the 24 pieces of information about the amount of received light ΔAₙ(λ₁), ΔAₙ(λ₂) and ΔAₙ(λ₃) that have been stored in the memory 25.

The measurement data display control unit 40 carries out such control that measurement data Xₙ(t), measurement data Yₙ(t) and measurement data Zₙ(t) are displayed on the measurement related points Mₙ and Sₙ in the three-dimensional head surface image 41 and in the three-dimensional brain surface image 42 on the basis of the measurement data Xₙ(t), Yₙ(t) and Zₙ(t) calculated by the operation unit 22, the channel information stored in the memory 25 and the placed point information prepared by the placed point positional relationship acquisition unit 37 when a doctor, a clinical examination technician or the like uses the input unit 27 to input an operation signal for displaying the measurement data Xₙ(t), Yₙ(t) and Zₙ(t)(n=1, 2 . . . , 24).

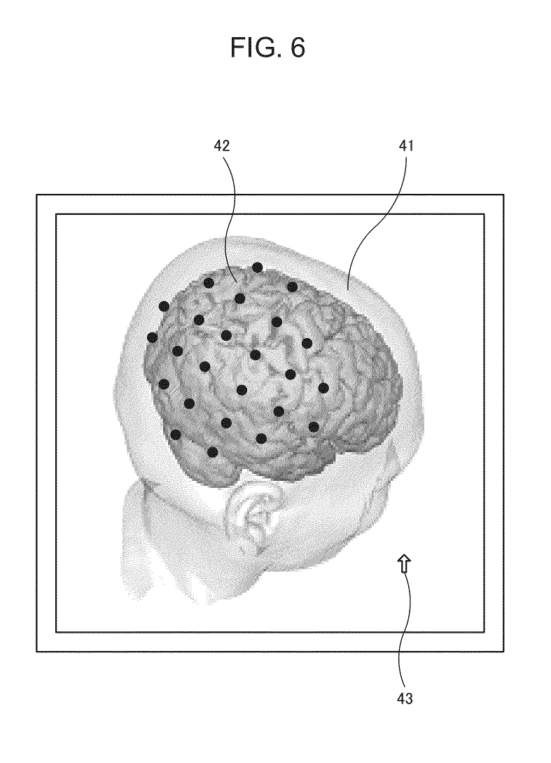

In the case where a doctor, a clinical examination technician or the like uses the input unit 27 to give instructions to the image switching unit 32c so as to display the three-dimensional head surface image 41 and the three-dimensional brain surface image 42 and so as to display measurement data Xₙ(t₁) at a certain point in time t₁, for example, the measurement data Xₙ(t₁) at a certain point in time t₁ is displayed on the three-dimensional head surface image 41 by preparing color mapping through determining a color on the basis of the color table showing the correspondence between numeric values and colors and through calculating the measurement relating point Mₙ (n=1, 2 . . . , 24) on the three-dimensional head surface image 41 (see FIG. 6).

At this time, the 24 measurement related points Mₙ are calculated in such a manner that the measurement related point M₁ is the middle point of the line segment that connects the light sending probe point T1 and the light receiving probe point R1, and the measurement related point M₂ is the middle point of the line segment that connects the light sending probe point T2 and the light receiving probe point R1.

In addition, in the case where a doctor, a clinical examination technician or the like uses the input unit 27 to give instructions to the image switching unit 32c so as to display the three-dimensional brain surface image 42 and so as to display measurement data Yₙ(t₂) at a certain point in time t₂, the measurement data Yₙ(t₂) at a certain point in time t₂ is displayed on the three-dimensional brain surface image 42 by preparing color mapping through determining a color on the basis of the color table showing the correspondence between numeric values and colors and through calculating the measurement relating point Sₙ (n=1, 2 . . . , 24) on the three-dimensional brain surface image 42.

At this time, the 24 measurement related points Sₙ are calculated in such a manner that the measurement related point S₁ is located at a depth equal to half the distance between the light sending probe point T1 and the light receiving probe point R1 beneath the middle point M₁ of the line segment that connects the light sending probe point T1 and the light receiving probe point R1, and the measurement related point S₂ is located at a depth equal to half the distance between the light sending probe point T2 and the light receiving probe point R1 beneath the middle point M₂ of the line segment that connects the light sending probe point T2 and the light receiving probe point R1.

### Other Embodiments

(1) Though the above described optical biometric device 1 has such a configuration that the light sending probe points T1 through T8 are displayed as red globes and, at the same time, the light receiving probe points R1 through R8 are displayed as blue globes, the probe points may be represented as polygons or characters.

(2) Though the above described optical biometric device 1 has such a configuration that the channel information display control unit 39 displays a display screen as in FIG. 5 after information about the point locations has been prepared in the placed point positional relationship acquisition unit 37, the configuration may allow a display screen to be displayed whenever information about point locations is acquired in the placed point positional relationship acquisition unit 37. As a result, points and lines are displayed whenever designation is made using the stylus 15.

### INDUSTRIAL APPLICABILITY

The present invention can be applied to an optical biometric device or the like for non-invasively measuring brain activity.

### EXPLANATION OF SYMBOLS

1: optical biometric device
12: light sending probe
13: light receiving probe
21: control unit for sending and receiving light
22: operation unit
25: memory (storage unit)
26: display unit
30: holder (light sending/receiving unit)
32: three-dimensional image display control unit
39: channel information display control unit
40: measurement data display control unit
41: three-dimensional head surface image
42: three-dimensional brain surface image

1. An optical biometric device, comprising:
a light sending/receiving unit having a number of light sending probes to be placed on a surface of the head of a subject and a number of light receiving probes to be placed on a surface of the head;
a control unit for sending and receiving light which acquires a number of pieces of information about the amount of light received in a number of measurement portions under control such that said light sending probes irradiate the surface of the head with light, and at the same time said light receiving probes detect light emitted from the surface of the head;
an operation unit for acquiring a number of pieces of measurement data on the basis of a number of pieces of information about the amount of received light;
a three-dimensional image display control unit for acquiring a three-dimensional head surface image and a three-dimensional brain surface image and displaying the acquired images on a display unit; and
a measurement data display control unit for displaying a number of pieces of measurement data on the three-dimensional head surface image or three-dimensional brain surface image displayed on the display unit, characterized by further comprising:
a storage unit for storing channel information that indicates combinations of light sending probes and light receiving probes for acquiring information about the amounts of the received light in measurement portions; and
a channel information display control unit for displaying a number of light sending probe points at which light sending probes have been placed and a number of light receiving probe points at which a number of light receiving probes have been placed according to the three-dimensional coordinates displayed on the display unit, and at the same time, for displaying line segments that indicate combinations of light sending probes and light receiving probes for connecting light sending probe points and light receiving probe points based on said channel information.

2. The optical biometric device according to claim 1, characterized by further comprising:
a magnetic field source for generating a magnetic field in a space including and surrounding the head of said subject that is fixed to a set point on the head of said subject;
a magnetic sensor for designation that detects a magnetic field in order to designate a point on the surface of the head of said subject;
a standard positional relationship acquisition unit for acquiring the positional relationship between said magnetic field source and at least three standard points by gaining a detection signal from said magnetic sensor for designation when the three standard points are designated on the surface of the head of said subject by the magnetic sensor for designation;
a correspondence data preparation unit for preparing correspondence data that indicates the correspondence between the three standard points and at least three standard point images when the three standard point images are designated on said three-dimensional head surface image by an input unit; and
a placed point positional relationship acquisition unit for acquiring positional relationships between said magnetic source and the points at which the light sending probes and the light receiving probes are placed by gaining a detection signal from said magnetic sensor for designation when the points at which the light sending probes are placed and the points at which the light receiving probes are placed on the surface of the head of said subject are designated by the magnetic sensor for designation.

3. A position measuring device used in an optical biometric device comprising:
a light sending/receiving unit having a number of light sending probes to be placed on a surface of the head of a subject and a number of light receiving probes to be placed on a surface of the head; and
a control unit for sending and receiving light which acquires a number of pieces of information about the amount of light received in a number of measurement portions under control such that said light sending probes irradiate the surface of the head with light, and at the same time said light receiving probes detect light emitted from the surface of the head, characterized by comprising:
a storage unit for storing channel information that indicates combinations of light sending probes and light receiving probes for acquiring information about the amounts of the received light in measurement portions; and
a channel information display control unit for displaying a number of light sending probe points at which light sending probes have been placed and a number of light receiving probe points at which a number of light receiving probes have been placed according to the three-dimensional coordinates displayed on a display unit, and at the same time, for displaying line segments that indicate combinations of light sending probes and light receiving probes for connecting light sending probe points and light receiving probe points based on said channel information.
